(19) United States
(12) Patent Application Publication
(10) Pub. No.: US 2019/0066833 A1
(43) Pub. Date: Feb. 28, 2019

(54) SYSTEM AND METHOD FOR MONITORING PHYSICAL THERAPY

(71) Applicant: Arctos Medical Technologies, Denver, CO (US)

(72) Inventor: Benjamin Wicki, Denver, CO (US)

(73) Assignee: Arctos Medical Technologies, Denver, CO (US)

(21) Appl. No.: 16/103,365

(22) Filed: Aug. 14, 2018

Related U.S. Application Data
(60) Provisional application No. 62/549,388, filed on Aug. 23, 2017.

(51) Int. Cl. G16H 20/30; G16H 80/00

(52) U.S. Cl. CPC G16H 20/30; A63B 21/00047; A63B 2220/833; G16H 80/00

(57) ABSTRACT

Various embodiments of the invention are directed to a system and method of providing physical therapy to a patient. In another embodiment, the present disclosure pertains to a method and system for providing physical therapy to a patient whereby the treatment is part of a pre-surgery requirement. In another embodiment, the present disclosure is directed to a method and system for providing physical therapy to a patient whereby the treatment is part of post-surgical care. Various embodiments incorporate a physical therapy application communicatively connected to a physical therapy unit configured to transmit, receive and store data related to physical therapy care.

### CROSS-REFERENCE TO RELATED APPLICATIONS

This application claims priority to U.S. Provisional Patent Application 62/549,388, filed on Aug. 23, 2017, which is incorporated in its entirety herein.

### FIELD OF THE INVENTION

The present disclosure relates to systems and methods for monitoring and tracking performance of physical therapy activities and for evaluating progress in performing a physical therapy routine.

### BACKGROUND OF THE INVENTION

Physical therapy procedure has changed very little since the days of Per Henrik Ling, the apparent “Father of Swedish Gymnastics,” who founded the Royal Central Institute of Gymnastics (RCIG) in 1813. Pursuant to this procedure, a patient is referred by a doctor to a physical therapist as a result of some injury or post orthopedic surgery. After a brief consultation with the patient, the physical therapist makes an initial treatment plan. Therapy will generally include useful manipulation of the injured part to do either at the therapist's office or at the patient's home. The patient is typically required to self-monitor his or her own progress and treatment is finished when the patient achieves his or her functional goal or funds run out. This procedure is particularly problematic when the therapy is part of a pre or post-surgical requirement or when the therapy is funded by a third-party vehicle such as a worker's compensation policy.

The potential for abuse of this type of arrangement has created a need for objective, computerized monitoring of the physical therapy actually performed by or for a patient and of the patient's progress in completing a physical therapy regimen.

### SUMMARY

In one embodiment, the present disclosure relates to a method and system for providing physical therapy to a patient. In various embodiments, such provision of therapy takes place whereby the treatment is being paid for by the patient's employer, such as via a workers compensation policy. The patient creates an account providing demographical and biometric information such as name, age, language, height, weight, body-mass index (BMI), blood pressure. In some instances, this information will be imported directly from the patient's electronic medical files. The patient downloads the physical therapy application (PTA) onto a compatible device such as a smartphone and is provided the appropriate physical therapy unit (PTU) for their size and injured body part. The PTU is tested and calibrated on an individual basis for the patient and baseline data is taken and stored in memory. The patient's medical prescription is translated into an appropriate treatment with the PTU and the treatment is uploaded to the PTA. During treatment, the PTU and PTA communicate wirelessly and record and store the patient's progress. The patient's medical team and other approved parties can be provided access to the progress of patient's treatment. Patient's treatment program can be altered as appropriate during treatment by authorized users of the PTA. The patient can be rewarded or penalized per the workers compensation policy terms at set intervals pending compliance and agreed upon metrics.

In another embodiment, the present disclosure pertains to a method and system for providing physical therapy to a patient whereby the treatment is part of a pre-surgery requirement. The patient creates an account providing demographical and biometric information such as name, age, language, height, weight, body-mass index (BMI), blood pressure. In some instances, this information will be imported directly from the patient's electronic medical files. The patient downloads the physical therapy application (PTA) onto a compatible device such as a smartphone and is provided the appropriate physical therapy unit (PTU) for their size and anticipated surgery. The PTU is tested and calibrated on an individual basis for the patient and baseline data is taken and stored in memory. The patient's medical prescription for pre-surgery care is translated into an appropriate treatment with the PTU and the treatment is uploaded to the PTA. During treatment, the PTU and PTA communicate wirelessly and record and store the patient's progress. The patient's medical team and other approved parties can be provided access to the progress of patient's treatment. Patient's treatment program can be altered as appropriate during treatment by authorized users of the PTA. Upon completion, the patient's medical team can review the results and assess the appropriate surgical or conservative intervention.

In another embodiment, the present disclosure is directed to a method and system for providing physical therapy to a patient whereby the treatment is part of post-surgical care. The patient creates an account providing demographical and biometric information such as name, age, language, height, weight, body-mass index (BMI), resting blood pressure, resting heart rate. In some instances, this information will be imported directly from the patient's electronic medical files. The patient downloads the physical therapy application (PTA) onto a compatible device such as a smartphone and is provided the appropriate physical therapy unit (PTU) for their size and anticipated surgery. The PTU is tested and calibrated on an individual basis for the patient and baseline data is taken and stored in memory. The patient's medical prescription for post-surgical care is translated into an appropriate treatment with the PTU and the treatment is uploaded to the PTA. During treatment, the PTU and PTA communicate wirelessly and record and store the patient's progress. The patient's medical team and other approved parties can be provided access to the progress of patient's treatment. Patient's treatment program can be altered as appropriate during treatment by authorized users of the PTA. Upon completion, the patient's medical team can review the results and assess the patient's progress in post-surgical rehabilitation.

### BRIEF DESCRIPTION OF THE FIGURES

The invention is more fully appreciated in connection with the following detailed description taken in conjunction with the accompanying drawings, in which:

FIG. 1A is a diagram illustrating a system for monitoring physical therapy performed by a patient.

FIG. 1B illustrates a physical therapy unit (PTU) placed under the back of a patient performing physical therapy.

FIG. 2 is a flowchart illustrating an example process for monitoring a patient's performance of physical therapy in accordance with an embodiment.

FIG. 3 is a flowchart illustrating an example process for monitoring a patient receiving treatment for pre-surgery care.

FIG. 4 is a flowchart illustrating an example process for monitoring a patient receiving treatment for post-surgery care.

Like reference numerals refer to corresponding parts throughout the several views of the drawings.

### DETAILED DESCRIPTION

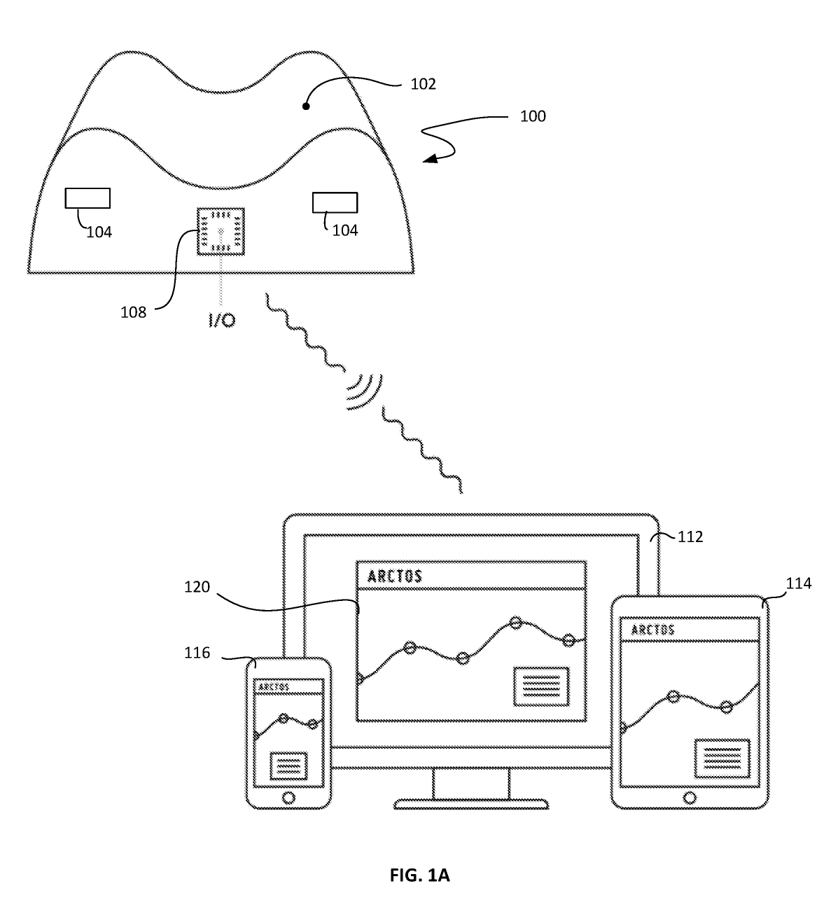

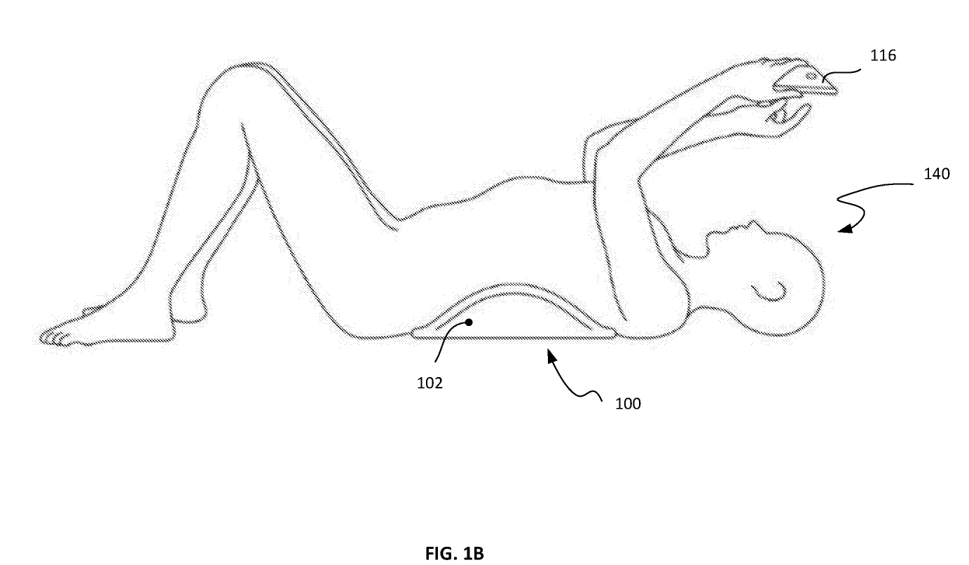

Disclosed herein is a physical therapy unit (PTU) tailored for a specific therapy to be administered and methods of utilizing such a PTU to monitor and assess a patient's performance of physical therapy. Examples of PTUs include back pillows, neck pillows, knee straps, sleeves, and shoulder straps. The PTU can be worn on, laid on, or affixed to the body during physical therapy. PTUs incorporate various electronic sensors or other measurement devices designed to capture comprehensive data with respect to physical activity, including but not limited to: time, date, duration, location, pressure, physical strength, speed, rotation, the placement and alignment of body parts during therapy activities. Specific sensors that may be considered include: inertial measurement units, pressure sensors, spatial sensors, gyroscopes, and accelerometers. PTUs can also incorporate antimicrobial coating for decreased infection and disease spread. The PTUs physical design will vary according to the specific treatment protocols and areas treated (knee vs. lower back, for example). In a specific embodiment of the invention, the PTU comprises a lower back pillow incorporating embedded electronic sensors. In such embodiment, the sensors collect data with respect to physical activity and then transmit such data to software operating on a mobile device, including but not limited to a tablet, phone, or computer. In a specific embodiment, the external surface of the PTU is comprised primarily of a breathable mesh. In an embodiment, the height of such PTU is approximately 4.5 inches, the width is approximately 13.75 inches, and the length is approximately 13.5 inches configured as depicted in FIGS. 1A and 1B. In an embodiment, the configuration of the PTU 102 is such that it corresponds to the contours of the lower back of the human body as depicted in FIG. 1B.

The PTU may wirelessly communicate with a physical therapy application (PTA), which may be installed on a device such as a handheld device. The PTA receives and stores the data generated by the PTU. For example, the PTA can receive data from PTU sensors which quantify the strength of a patient performing a therapeutic exercise such as an isometric contraction. The PTA can also provide the patient with real-time feedback on the accuracy of the therapeutic exercise which can be delivered visually or audibly. Feedback can also be rendered using virtual reality technology as known by those skilled in the art to allow the patient more freedom to move without the constraint of holding the device running the PTA.

In one embodiment the PTA software is designed to track compliance and time spent using a PTU by measuring whether the patient did the activities at the specified times/dates for the correct duration, frequency, and sequence. By capturing and recording data outputs from the PTUs measurement focused sensors embedded in the PTU, it is possible to analyze and assess how well the patient performed the prescribed activities and not just whether the activities were performed at all. The PTA can further capture subjective information provided by the patient or other participants in the treatment regimen (including self-assessments regarding health, well-being and the treatment milestones). The PTA may include software that analyzes the results of activity and the patient's interaction with the PTU and then communicates that information to the appropriate medical professionals and other participants, including the patient; and any approved third-party. The PTA software also facilitates the ability of medical professionals, the patient and other participants in the treatment process to continuously monitor and change the treatment protocols to achieve the desired results.

In one exemplary embodiment, the PTU measures abdominal isometric strength and control during an isometric abdominal contraction with integrated inertial, location and navigation sensors. These measurements are captured and sent to the PTA, informing a healthcare provider about compliance, abdominal strength, and abdominal isometric endurance control of the patient. The PTA software can also progress the patient through established treatment protocols to improve technique and performance using an interactive design that facilitates compliance. The PTA software is optimally an installed application based program that does not require continuous access to the internet, however, the PTA can also be delivered via the cloud as a software as a service (SaaS) program that may be purchased, license or subscribed to.

The PTU and PTA communicate wirelessly using wireless protocols including, but not limited to, RF, Bluetooth, 802.1, CDMA, CDMA2000, LTE, or any other wireless technology. The PTA software enables presentation and communication of performance data in real time as well as storage for subsequent consideration and analysis by the patient, the patient's medical team, or other approved third parties.

In another embodiment, the present disclosure is directed to a method and system for providing physical therapy to a patient whereby the treatment is being paid for by the patient's employee such as via a workers compensation policy. These policies often have requirements attached such that the patient must follow a certain prescribed therapy regimen. The patient creates an account providing demographical and biometric information such as name, age, language, height, weight, body-mass index (BMI), blood pressure. In some instances, this information will be imported directly from the patient's electronic medical files. The patient downloads the physical therapy application (PTA) onto a compatible device such as a smartphone or tablet and is provided the appropriate physical therapy unit (PTU) for their size and injured body part. The PTU is tested and calibrated on an individual basis for the patient and baseline data is taken and stored in memory. The patient's medical prescription is translated into an appropriate treatment with the PTU and the treatment is uploaded to the PTA. During treatment, the PTU and PTA communicate wirelessly and record and store the patient's progress. The patient's medical team and other parties can be provided access to the progress of patient's treatment. The patient can also access a help-line to assist in use of the PTA and PTU, as well as to report on issues with the prescribed treatment. Patient's treatment program can also be altered as appropriate during treatment by authorized users of the PTA. Upon completion of the treatment, the patient can be rewarded or penalized per the workers compensation policy terms at set intervals pending compliance and agreed upon metrics. Additionally, non-compliance can be reported to involved third-parties such as the patient's medical team or the patient's employer. This use can reduce cost for insurance companies by limiting medical expenditures while maximizing patient outcomes, reduce the cost for employers by minimizing lost productivity, medical compensation and time of leave, reduce the cost for workers compensation patients by minimizing lost wages, and reduce the cost for traditional patients by minimizing supplemental assistance, copayments, medical visits and associated costs.

In another embodiment, the present disclosure is directed to a method and system for a wellness program for at-risk employees as an injury prevention tool administered and monitored by a physical therapist. Upon completion of the treatment, the employee/patient would be rewarded or penalized per the pre-approved agreement with their employee at set intervals pending compliance and agreed upon metrics. This use can reduce the cost for the employers by minimizing lost productivity, medical compensation and time of leave, reduce the cost of workers compensation by minimizing lost wages.

In another embodiment, the present disclosure pertains to a method and system for providing physical therapy to a patient whereby the treatment is part of a pre-surgery requirement. This use aids in determining the potential efficacy of surgery for a patient and the likelihood of certain outcomes. Treatment can be personalized depending on treatment goals and surgical protocols based on pre and post-surgical data to maximize successful outcomes. The patient creates an account providing demographical and biometric information such as name, age, language, height, weight, body-mass index (BMI), blood pressure. In some instances, this information will be imported directly from the patient's electronic medical files. The patient downloads the physical therapy application (PTA) onto a compatible device such as a smartphone and is provided the appropriate physical therapy unit (PTU) for their size and anticipated surgery. The PTU is tested and calibrated on an individual basis for the patient and baseline data is taken and stored in memory. The patient's medical prescription for pre-surgery care is translated into an appropriate treatment with the PTU and the treatment is uploaded to the PTA. During treatment, the PTU and PTA communicate wirelessly and record and store the patient's progress. The patient's medical team and other parties can be provided access to the progress of patient's treatment. Patient's treatment program can be altered as appropriate during treatment by authorized users of the PTA. Upon completion, the patient's medical team can review the results and assess the appropriate surgical or conservative intervention.

In another embodiment, the disclosure is directed to a method and system for providing physical therapy to a patient whereby the treatment is part of post-surgical care. The patient creates an account providing demographical and biometric information such as name, age, language, height, weight, body-mass index (BMI), blood pressure. In some instances, this information will be imported directly from the patient's electronic medical files. The patient downloads the physical therapy application (PTA) onto a compatible device such as a smartphone and is provided the appropriate physical therapy unit (PTU) for their size and anticipated surgery. The PTU is tested and calibrated on an individual basis for the patient and baseline data is taken and stored in memory. The patient's medical prescription for post-surgical care is translated into an appropriate treatment with the PTU and the treatment is uploaded to the PTA. During treatment, the PTU and PTA communicate wirelessly and record and store the patient's progress. The patient's medical team and other parties can be provided access to the progress of patient's treatment. Patient's treatment program can be altered as appropriate during treatment by authorized users of the PTA. Upon completion, the patient's medical team can review the results and assess the patient's progress in post-surgical rehabilitation. This enables homebound patients or patients living in rural locations better access to medical care and limits the number of required medical visits.

FIG. 1A is a diagram illustrating a system for monitoring physical therapy performed by a patient. In this example, Physical Therapy Unit (PTU) 100 includes a pillow 102 designed for use in connection with a physical therapy protocol designed to, for example, rehabilitate a patient having an injured back. It may be appreciated that alternative pillow designs or structures may be used to facilitate rehabilitation of other body parts. In an embodiment, the covering of the pillow primarily comprises a breathable mesh fabric. In the embodiment of FIG. 1A, the PTU 100 includes one or more sensors 104 that have embedded circuitry and software associated with recording the physical activity done during physical therapy. In an embodiment, the one or more sensors comprise an inertial measurement unit, optionally a 6-axis inertial measurement unit comprised of an accelerometer and a gyroscope. In an embodiment, the PTU 100 incorporates a battery to power the one or more sensors, embedded circuitry and associated electronics in accordance with methods and mechanisms readily apparent to one skilled in the art. In an embodiment, the battery comprises a lithium ion battery. An input/output (10) unit 108 coupled to, or forming a part of, the one or more sensors 104 communicates wirelessly via one or more networks with a remote device such as, for example, a desktop computer 112, electronic tablet, 114, or handheld personal electronic device 116. A Physical Therapy Application 120 (PTA) installed on one or more of the remote devices 112, 114, 116 receives and processes data provided by the sensors 104 in order to, for example, track a patient's 140 compliance with a physical therapy treatment program or otherwise monitor a patient's 140 progress in following a rehabilitation regimen.

Turning now to FIG. 1B, an illustration is provided of one manner in which the PTU 100 may be placed under the back of a patient 140 performing physical therapy. As shown, in the embodiment of FIG. 1B the patient 140 is holding the personal electronic device 116 while performing a physical therapy routine. The patient 140 may receive instruction relating to performance of therapy through a user interface of the personal electronic device 116 (e.g., through a video presented via Web page, an app containing step-by-step instructions, and the like). The personal electronic device 116 may simultaneously receive information relating to the performance by the patient 140 of the physical therapy protocol and may transmit the same (either in real time or after the patient has completed a physical therapy session) to a remote server or cloud-based infrastructure over one or more networks (not shown).

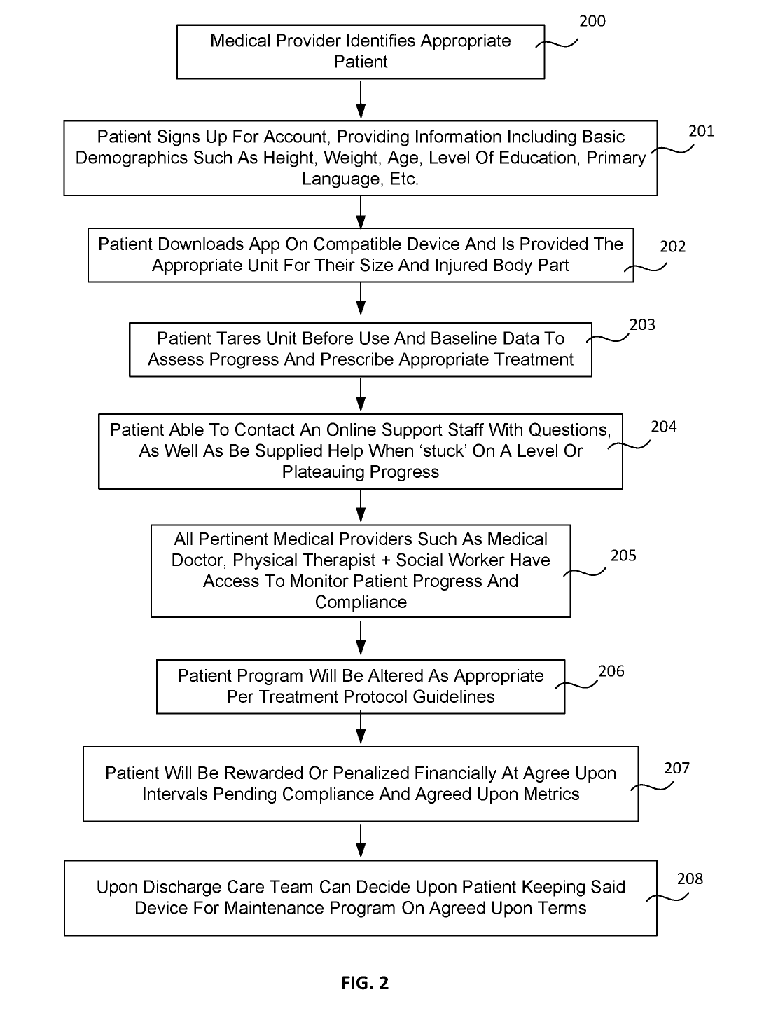

FIG. 2 is a flowchart illustrating an example process for monitoring a patient's performance of physical therapy in accordance with an embodiment. In the example of FIG. 2, the patient may be performing physical therapy as a condition of receiving financial compensation such as, for example, workers compensation. First, in step 200, the medical provider identifies the appropriate patient. In step 201, the patient signs up for an account providing demographical and biometric information such as name, age, level of education, language, height, weight, body-mass index (BMI), and blood pressure. In step 202, the patient downloads the PTA 120 onto a compatible device such as a smartphone 116. The patient is also provided the physical therapy unit PTU, such as the PTU 100. The PTU is appropriately selected in view of patient's size and injured body part. In step 203, the patient tests and calibrates the PTU and baseline data is taken and stored in memory. In step 204, the patient is able to contact online support staff with questions as well as being supplied with help when stuck on a therapy level. In step 205, the patient's medical team and other parties such as insurance companies and social workers are provided access to the progress and compliance of patient's treatment. In step 206, the patient's program is altered as appropriate per treatment protocol guidelines. In step 207, the patient is rewarded or penalized financially at agreed upon intervals pending compliance and agreed upon metrics. Lastly, in step 208, the patient is discharged and the discharge care team decides upon whether the patient keeps the PTU for maintenance on agreed upon terms.

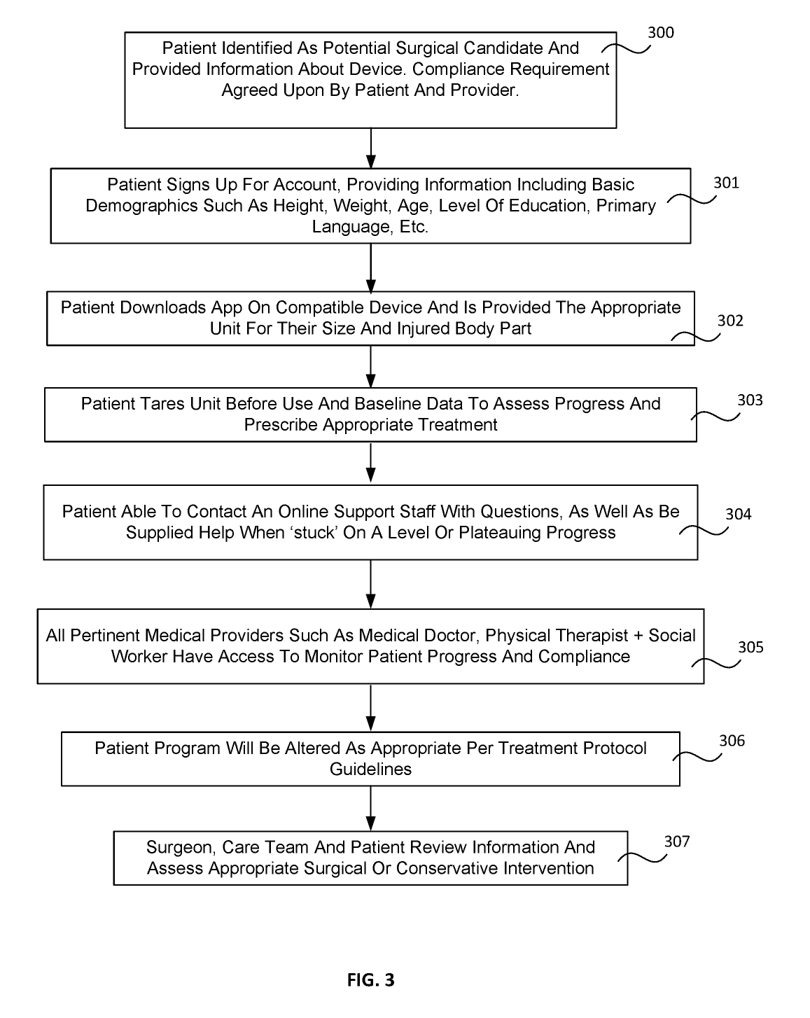

FIG. 3 is a flowchart illustrating an example process for monitoring a patient receiving treatment for pre-surgery care. First, in step 300, the patient is identified as a potential surgical candidate and is provided information about the PTU and compliance with the therapy is agreed upon by the patient and provider. In step 301, the patient signs up for an account providing demographical and biometric information such as name, age, level of education, language, height, weight, body-mass index (BMI), and blood pressure. In step 302, the patient downloads the physical therapy application (PTA) onto a compatible device such as a smartphone and is provided the appropriate physical therapy unit (PTU) for their size and injured body part. In step 303, the patient tests and calibrates the PTU and baseline data is taken and stored in memory. In step 304, the patient is able to contact online support staff with questions as well as being supplied with help when stuck on a therapy level. In step 305, the patient's medical team and other parties such as insurance companies and social workers are provided access to the progress and compliance of patient's treatment. In step 306, the patient's program is altered as appropriate per treatment protocol guidelines. Lastly, in step 307, the surgical team and patient review the therapy information and assess appropriate surgical or conservative intervention.

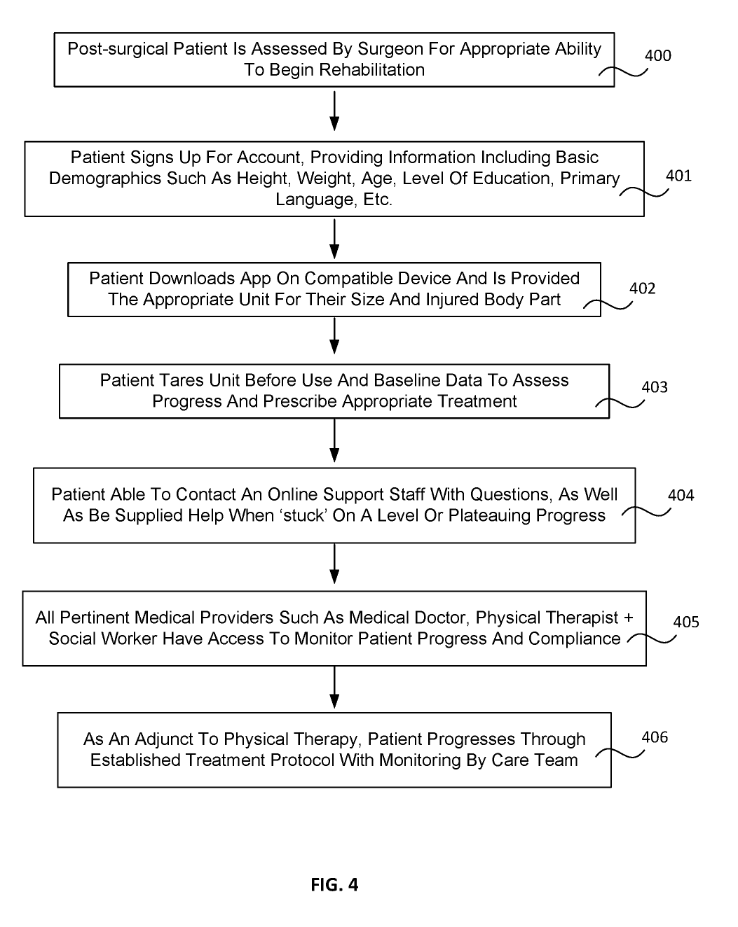

FIG. 4 is a flowchart illustrating an example process for monitoring a patient receiving treatment for post-surgery care. First, in step 400, the post-surgical patient is assessed by the surgical team for appropriate ability to begin any rehabilitation therapy using a PTU. In step 401, the patient signs up for an account providing demographical and biometric information such as name, age, level of education, language, height, weight, body-mass index (BMI), and blood pressure. In step 402, the patient downloads the physical therapy application (PTA) onto a compatible device such as a smartphone and is provided the appropriate physical therapy unit (PTU) for their size and injured body part. In step 403, the patient tests and calibrates the PTU and baseline data is taken and stored in memory. In step 404, the patient is able to contact online support staff with questions as well as being supplied with help when stuck on a therapy level. In step 405, the patient's medical team and other approved parties such as insurance companies and social workers are provided access to the progress and compliance of patient's treatment. Lastly, in step 406, the patient progresses through the established treatment protocol with monitoring by the patient's care team.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

What is claimed is:

1. A method for remotely monitoring performance of a physical therapy, the method comprising:
presenting, through a user interface of an electronic device, information relating to a physical therapy prescription;
receiving, at the electronic device, sensor data from a physical therapy unit configured to sense performance of a physical therapy activity by a user wherein the physical therapy activity is included within the physical therapy prescription and wherein the physical therapy unit is at least one of coupled to the user or in contact with a body part of the user;
transmitting, from the electronic device, monitoring information to one or more third parties wherein the monitoring information is based upon the sensor data.

2. The method of claim 1 further including receiving, at the electronic device, modifications to the physical therapy prescription and storing the modifications within a memory of the electronic device wherein the modifications are prescribed based upon the monitoring information.

3. The method of claim 1 wherein the sensor data quantify a strength of the user in performing the physical therapy activity.

4. The method of claim 1, further including providing feedback to the user via the user interface of the electronic device wherein the feedback relates to an extent to which the physical therapy activity is being accurately performed by the user.

5. The method of claim 1 wherein the physical therapy unit includes a pillow element to which are coupled one or more sensors.

6. A method for remotely monitoring physical performance of a user, the method comprising:
presenting, through a user interface of an electronic device, information relating to at least one of an exercise routine or instructions for muscle engagement;
receiving, at the electronic device, sensor data from a monitoring unit configured to sense at least one of utilization or performance of one or more muscle groups of the user while the user performs the exercise routine or follows the instructions for muscle engagement, the monitoring unit being at least one of coupled to the user or in contact with a body part of the user;
transmitting, from the electronic device, monitoring information to one or more third parties wherein the monitoring information is based upon the sensor data.

7. The method of claim 1 wherein the physical therapy prescription includes information relating to a plurality of physical activities for facilitating recovery from an injury sustained by the user.

8. The method of claim 1 wherein the physical therapy prescription includes information relating to a plurality of physical activities for enhancing physical performance of the user.

9. A pillow for use in association with physical therapy activities, comprising:
a battery, and
sensors,
the sensors configured to collect data with respect to physical activity, and
the sensors configured to transmit the data wirelessly to a remote device.

10. The pillow of claim 9, wherein at least one of the sensors consists of an inertial measurement unit.

11. The pillow of claim 10, wherein the inertial measurement unit comprises an accelerometer and a gyroscope.

12. The pillow of claim 9, wherein at least one of the sensors incorporates embedded circuitry and software configured to collect data summarizing the activity performed during physical therapy.

13. The pillow of claim 9, configured to correspond with the contours of a human's lower back.

14. The pillow of claim 9, further comprising a breathable mesh covering.
